(19) United States
(12) Patent Application Publication
(10) Pub. No.: US 2026/0263135 A1
(43) Pub. Date: Sep. 10, 2026

(54) VISUAL LIGHT SEALING CONFIRMATION IN CRYOBALLOON ABLATION CATHETERS

(71) Applicant: Medtronic Ireland Manufacturing Unlimited Company, Dublin (IE)

(72) Inventor: Nicolas Coulombe, Anjou (CA)

(21) Appl. No.: 19/552,905

(22) Filed: Feb. 27, 2026

Related U.S. Application Data
(60) Provisional application No. 63/766,674, filed on Mar. 4, 2025.

(51) Int. Cl. A61B 18/02; A61B 90/00; A61B 18/00

(52) U.S. Cl. CPC A61B 18/02; A61B 90/06; A61B 90/08; A61B 2018/00577; A61B 2018/00642; A61B 2018/00744; A61B 2018/00791; A61B 2018/0091; A61B 2018/0212; A61B 2018/0262; A61B 2090/064; A61B 2090/0807

(57) ABSTRACT

Systems and methods for operating cryoballoon ablation catheters. One example system includes a catheter including a treatment element including an expandable element, a light emitting device positioned and configured to emit light at the expandable element, and an electronic controller coupled to the catheter. The electronic controller is configured to initiate an ablation process by controlling a fluid source to provide fluid to the expandable element of the treatment element and selectively operate the light emitting device during the ablation process.

### RELATED APPLICATIONS

This application claims the benefit of U.S. Provisional Application No. 63/766,674 filed Mar. 4, 2025, the entire contents of which are hereby incorporated by reference.

### BACKGROUND

Cryotherapy is a useful treatment modality for many types of medical procedures. In some cases, it is desirable to administer cryotherapy from within a patient's body, such as from within a body lumen. Internal administration of cryotherapy can be advantageous, for example, in at least some bronchial procedures. These procedures can include percutaneously introducing a cryotherapeutic element into a patient and then advancing a catheter shaft carrying the cryotherapeutic element along an intravascular path to a suitable treatment location. Once positioned at the treatment location, the cryotherapeutic element can be cooled to modulate nearby nerves. The cooling caused by the cryotherapeutic element, for example, can reduce undesirable obstructions within the lumen.

### SUMMARY

Some endoscopic cyrotherapeutic catheter devices use cryoablation techniques. For example, in bronchoscopy a bronchoscope with a cryotherapeutic catheter (also known as a cryoprobe) may be used. During cryoablation treatment, a pressurized refrigerant is circulated through an occlusive balloon of the catheter, which has been inserted into a patient’s bronchus. The flow of refrigerant causes the occlusive balloon to expand within the bronchus. Adequate balloon expansion must be achieved and maintained to hold the occlusive balloon in place during the treatment, thus ensuring complete occlusion of the bronchus and creation of a circumferential lesion at the treatment site.

In some instances, it may be difficult for a user to visually determine whether total circumferential contact of the balloon with the bronchus has been achieved. For example, although the catheter device may include a light source and/or a camera to be used within the lumen as a visual aid, it may be difficult to position either or both of the light and the camera within the lumen to capture a desired view of the balloon. Fluid accumulation within the lumen may also obstruct a user’s view of the balloon.

Accordingly, embodiments and aspects described herein provide, among other things, catheters with treatment elements that include one or more light-emitting sources/devices (for example, light-emitting diodes (LEDs) or optical fiber). The catheter is configured to operate the LEDs as a visual aid (for example, during an ablation procedure) to help a user determine whether the total circumference of the balloon is in contact within the patient’s lumen (for example, as described in the example above, a bronchus).

In some aspects, the techniques described herein relate to a system for ablating tissue, the system including: a catheter including a treatment element, the treatment element including an expandable element; a light emitting device positioned and configured to emit light at the expandable element; and an electronic controller coupled to the catheter and configured to: initiate an ablation process by controlling a fluid source to provide fluid to the expandable element of the treatment element; and selectively operate the LED during the ablation process.

In some aspects, the techniques described herein relate to a method for operating an ablation catheter including a treatment element including an expandable element and a light emitting device positioned and configured to emit light at the expandable element, the method including: initiating an ablation process by controlling a fluid source to provide fluid to the expandable element of the treatment element; and selectively operate the light emitting device during the ablation process.

The details of one or more aspects of the disclosure are set forth in the accompanying drawings and the description below. Other features, objects, and advantages of the techniques described in this disclosure will be apparent from the description and drawings, and from the claims.

### BRIEF DESCRIPTION OF THE DRAWINGS

The accompanying figures, where like reference numerals refer to identical or functionally similar elements throughout the separate views, together with the detailed description below, are incorporated in and form part of the specification, and serve to further illustrate embodiments, examples, aspects, and features of concepts that include the claimed subject matter and explain various principles and advantages of those embodiments, examples, aspects, and features.

FIG. 1 is schematic illustration of a catheter system according to some examples.

FIG. 2 illustrates an inflow lumen of the system of FIG. 1 according to some examples.

FIG. 3 illustrates a treatment element of the balloon-based denervation system of FIG. 1 according to some examples.

FIG. 4 is a block diagram that illustrates an electronic controller of the system of FIG. 1 according to some examples.

FIG. 5A illustrates an expandable element of the treatment element of FIG. 3 disposed within a lumen according to some examples.

FIG. 5B illustrates an expandable element of the treatment element of FIG. 3 disposed within a lumen according to some examples.

FIG. 5C illustrates an expandable element of the treatment element of FIG. 3 disposed within a lumen according to some examples.

FIG. 5D illustrates an expandable element of the treatment element of FIG. 3 disposed within a lumen according to some examples.

FIG. 6 is a flowchart illustrating a method for operating the system of FIG. 1 according to some examples.

FIG. 7 is a flowchart illustrating a method for operating an LED of the system of FIG. 1 according to some examples.

Skilled artisans will appreciate that elements in the figures are illustrated for simplicity and clarity and have not necessarily been drawn to scale. For example, the dimensions of some of the elements in the figures may be exaggerated relative to other elements to help to improve understanding of examples, aspects, and features illustrated.

For ease of description, some or all of the example systems presented herein are illustrated with a single exemplar of each of its component parts. Some examples may not describe or illustrate all components of the systems. Other example implementations may include more or fewer of each of the illustrated components, may combine some components, or may include additional or alternative components.

In some instances, the apparatus and method components have been represented where appropriate by conventional symbols in the drawings, showing only those specific details that are pertinent to understanding the of various embodiments, examples, aspects, and features so as not to obscure the disclosure with details that will be readily apparent to those of ordinary skill in the art having the benefit of the description herein.

### DETAILED DESCRIPTION

Specific embodiments of the present disclosure are now described with reference to the figures, wherein like reference numbers indicate identical or functionally similar elements. The terms “distal” and “proximal” are used in the following description with respect to a position or direction relative to a surgical robot. “Distal” or “distally” are a position distant from or in a direction away from the surgical robot toward the patient. “Proximal” and “proximally” are a position near or in a direction away from the patient toward the surgical robot.

Before any examples are explained in detail, it is to be understood that the examples presented herein are not limited in their application to the details of construction and the arrangement of components set forth in the following description or illustrated in the following drawings. The examples are capable of other embodiments and of being practiced or of being carried out in various ways. For ease of description, the example systems presented herein may be illustrated with a single exemplar of each of its component parts. Some examples may not describe or illustrate all components of the systems. Other example embodiments may include more or fewer of each of the illustrated components, may combine some components, or may include additional or alternative components.

It should be understood that although certain figures presented herein illustrate hardware and software located within particular devices, these depictions are for illustrative purposes only. In some embodiments, the illustrated components may be combined or divided into separate software, firmware, and/or hardware. For example, instead of being located within and performed by a single electronic processor, logic and processing may be distributed among multiple electronic processors. Regardless of how they are combined or divided, hardware and software components may be located on the same computing device or may be distributed among different computing devices connected by one or more networks or other suitable communication links.

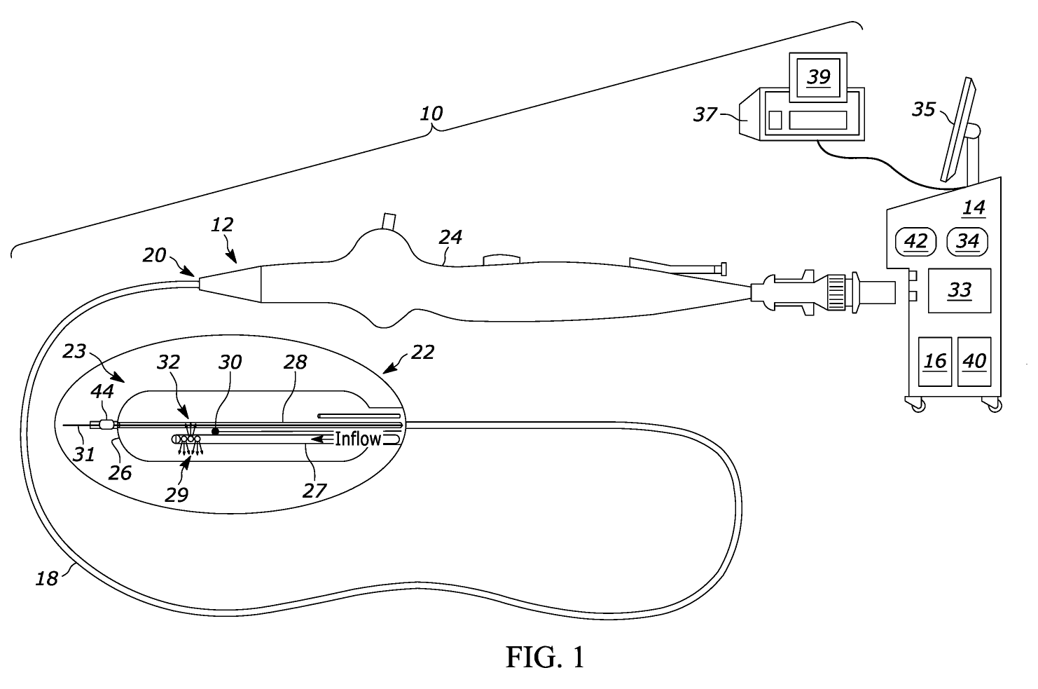

FIG. 1 illustrates an example system 10 that is suitable for performing cryoballoon ablation The system 10 may be suitable for performing endoscopic procedures including, for example, gallbladder, bladder, prostate, urethra, female reproductive system, and/orbronchial cryotherapy. The system 10 is for delivering thermally conductive fluid (refrigerant) to an area of target tissue, such as, for example, an area of tissue within a bronchus. The system 10 may generally include a catheter, such as a catheter 12, and a console 14 for operating, monitoring, and regulating the operation of the catheter 12 (for example, with the electronic controller 33), and a fluid source 16 for delivering fluid (for example, a refrigerant) to the catheter 12.

The catheter 12 is a highly flexible treatment device that is suitable for passage through the vasculature. The catheter 12 may be adapted for use with the fluid source 16 to denervate portions of a lumen (for example, a bronchus). In the example illustrated, the catheter 12 has an elongate body 18 having a proximal portion 20 and a distal portion 22. The distal portion 22 includes a treatment element 23. The proximal portion 20 of the catheter 12 is mated to a handle 24 that can include an element such as a lever or knob for manipulating the elongate body 18 and the treatment element 23. The distal portion 22 may also include an aperture (not shown) sized to allow for the passage of a guidewire 31 through the elongate body 18 and through the aperture.

The elongate body 18 is sized and configured to be passable through a patient’s lumen and/or positionable proximate to the area of target tissue, and may include one or more lumens (for example, the inflow lumen 27 and the inner member/guidewire lumen 28) disposed within the elongate body 18 that provide mechanical, electrical, and/or fluid communication between the proximal portion 20 of the elongate body 18 and the distal portion 22 of the elongate body 18. In some aspects, the elongate body 18 includes a guidewire lumen through which a sensing device, mapping device, the guidewire 31, or other system components may be located and extended from the distal portion 22 of the catheter 12. The elongate body 18 may be rigid and/or flexible to facilitate the navigation of the catheter 12 within a patient’s body. In one aspect, the distal portion 22 of the elongate body 18 is flexible to allow for more desirable positioning proximate to an area of target tissue (e.g., positioning within a bronchus or other lumen of the patient’s body).

The treatment element 23 includes an expandable element 26 (for example, an occlusive balloon), through which fluid from the fluid source 16 is circulated. An inflow lumen 27 is in fluid communication with the fluid source 16 in the console 14 to supply a refrigerant fluid (e.g., nitrous oxide (N₂O), nitrogen, carbon dioxide, argon, or another suitable refrigerant) in response to console commands and other control input. In some aspects, a vacuum pump 34 (electronically coupled to and controlled by the electronic controller 33) in the console 14 creates a low pressure environment in an outflow lumen (not shown) so that the fluid is drawn into the outflow lumen, away from the expandable element 26, towards the proximal portion 20 of the elongate body 18, and into the fluid recovery reservoir 40 within the console 14. In some aspects, the electronic controller 33 controls a valve or valves (not shown) to open and close to control the flow of a refrigerant fluid (e.g., stored under pressure in the fluid source 16) toward the treatment element 23. When expanded, the expandable element 26 is sized and configured to fit within an area of the lumen under treatment such that the treatment element 32 is substantially centered within the lumen. In some aspects, the expandable element 26 is composed of a translucent or transparent flexible material.

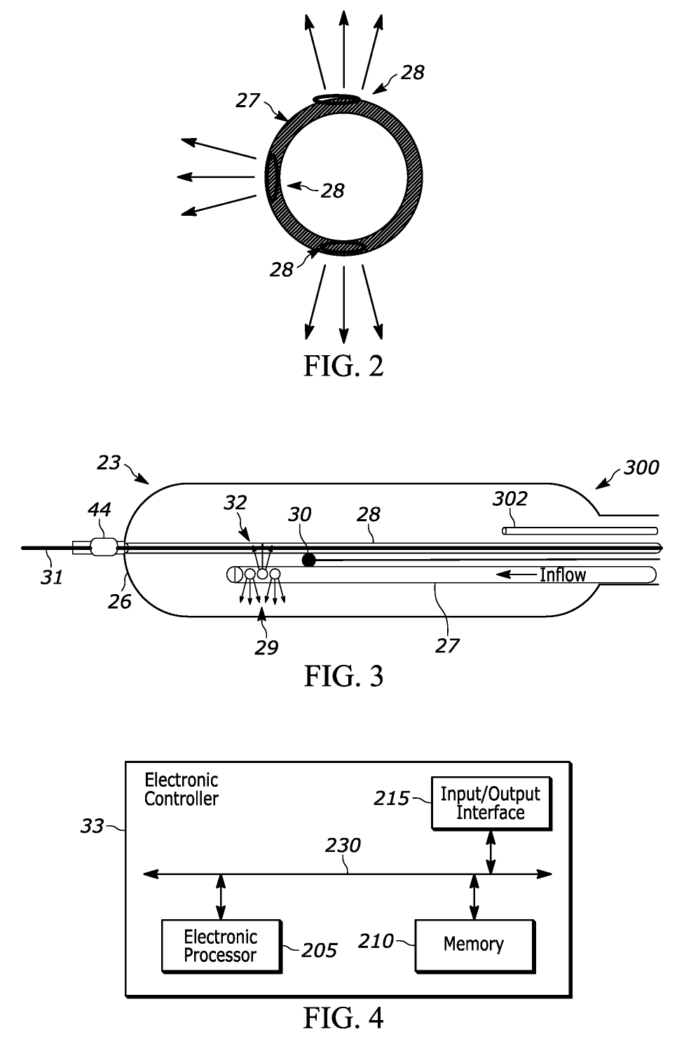

The inflow lumen 27, in some aspects, includes a plurality of throttle holes 29, which are disposed near the terminus of the inflow lumen 27. The throttle holes are configured to maximize circumferential ablation and heat transfer by producing a desired pressure drop. In some instances, the lumen 27 has between three and nine throttle holes. As illustrated in FIG. 2, the throttle holes may be distributed around the circumference of the inflow lumen 27. Returning to FIG. 1, the inflow lumen 27, in some aspects, is positioned such that it terminates proximate to the distal end of the expandable element 26 (for example, to improve thermal transfer from the target tissue to the refrigerant).

The treatment element 23 also includes a temperature sensor 30 (e.g., a thermocouple, a thermistor, a fiber optic temperature sensor, or another suitable means of sensing temperature), which is positioned within the expandable element 26 to measure the temperature of the fluid within the expandable element 26. The temperature sensor 30 is coupled to the electronic controller 33 of the console 14. In some aspects, the temperature sensor 30 is directly coupled to the electronic controller 33, which generates temperature values from voltages or other signals read from or provided by the temperature sensor 30. In other aspects, the temperature sensor 30 is indirectly coupled to the electronic controller 33 through, for example, intervening circuitry in the catheter 12 and the electronic controller 33 receives temperature values from the intervening circuitry.

In some aspects, the console 14 includes one or more pressure sensors 42 to continuously record the instantaneous pressure values within the expandable element 26. The pressure sensors 42 may then generate and transmit a pressure signal to the electronic controller 33 of the console 14. As illustrated in FIG. 3, in some aspects, treatment element 23 also includes a pressure monitoring tube 302 (enclosed in the elongate body 18) in fluid communication with the pressure sensor 42 (housed in the console 14) and the expandable element 26.

The catheter 12 includes one or more of a light emitting source/device. In the illustrated example, the catheter 12 includes one or more of a light emitting diode (LED) 44. In some aspects, the light emitting devices alternatively or additionally include a fiber optic light source. For ease of description, the light emitting source/device is described herein in terms of the LED 44. It should be understood that, in some aspects, other kinds of light emitting sources/devices may alternatively or additionally be utilized similarly in the system 10. In some aspects (for example, as illustrated in FIG. 1) at least one of the LED 44 is positioned at the distal portion 22 at a distal end of the expandable element 26. For example, the LED 44 is positioned between the guidewire 31 and the expandable element 26 in some aspects. Alternatively or additionally, in some aspects, the LED 44 is positioned between the expandable element 26 and the proximal end 20 of the catheter 12 (for example, on the elongate body 18 and/or on the handle 24 of the catheter 12). In some aspects, the LED 44 is positioned within the expandable element 26. For ease of description, the one or more LEDs 44 are described herein in terms of a single LED 44. It should be understood, however, that in some aspects more than one LED 44 may be included on the catheter 12. In such aspects, the electronic controller 33 is configured to operate any one of the LED 44 (for example, based on sensor information (for example, from the temperature sensor 30 and/or the pressure sensor(s) 42) and/or a user input received by the electronic controller 33 via input/output interface 215 described in more detail below with respect to FIG. 4). The LED 44, in some aspects, is positioned and configured such that a light output from the LED 44 is aimed at the expandable element 26.

In the illustrated example, the control unit 14 includes an electronic controller 33 (described more particularly with respect to FIG. 4) programmed or programmable to execute the automated or semi-automated operation and performance of the features, sequences, calculations, or procedures described herein. The electronic controller 33 is communicatively coupled to the various components of the catheter 12, including the vacuum pump 34, one or move valves as described herein, the temperature sensor 30, the pressure sensors 42, and the one or more LEDs. The control unit 14 may include one or more user input devices, controllers, speakers, and/or electronic displays 35 (each coupled to and controllable by the electronic controller 33) for collecting and conveying information from and to the user.

In some aspects, the treatment element 23 includes ultrasonic transducers (not shown) to record the reflected, refracted, scattered, and/or attenuated ultrasound signals from the target tissue. As the transducers record the ultrasound signals, they begin to vibrate and the mechanical vibrations are converted into electric current signals that are transmitted back to the control unit 14 or an external ultrasound control unit 37 which processes the signals to generate a sonogram or ultrasonogram showing the patient’s tissue, organs, and/or a location of the catheter 12 within the patient’s body. In some instances, the sonogram also includes other matter at or near the location of the catheter 12 within the patient’s body (for example, ice formations formed via the cryoablation procedure. The sonogram may then be relayed to a clinician via a display 35 of the control unit 14, or via a display 39 of the external ultrasound control unit 37, to assist the physician in positioning the catheter 12 near or proximate to a desired treatment location.

FIG. 4 illustrates an example embodiment of the electronic controller 33, which includes an electronic processor 205 (for example, a microprocessor, application specific integrated circuit, etc.), a memory 210, and an input/output interface 215. The memory 210 may be made up of one or more non-transitory computer-readable media and includes at least a program storage area and a data storage area. The program storage area and the data storage area can include combinations of several types of memory, such as read-only memory (“ROM”), random access memory (“RAM”) (for example, dynamic RAM (“DRAM”), synchronous DRAM (“SDRAM”), etc.), electrically erasable programmable read-only memory (“EEPROM”), flash memory, or other suitable memory devices. The electronic processor 205 is coupled to the memory 210 and the input/output interface 215. The electronic processor 205 sends and receives information (for example, from the memory 210 and/or the input/output interface 215) and processes the information by executing one or more software instructions or modules, capable of being stored in the memory 210, or another non-transitory computer readable medium. The software can include firmware, one or more applications, program data, filters, rules, one or more program modules, and other executable instructions. The electronic processor 205 is configured to retrieve from the memory 210 and execute, among other things, software for performing methods as described herein.

The input/output interface 215 transmits and receives information from devices external to the electronic controller 33 (for example, over one or more wired and/or wireless connections), for example, components of the system 10. The input/output interface 215 receives input (for example, from a human machine interface of the console 14), provides system output or a combination of both. The input/output interface 215 may also include other input and output mechanisms, which for brevity are not described herein and which may be implemented in hardware, software, or a combination of both.

It should be understood that although FIG. 4 illustrates only a single electronic processor 205, memory 210, and input/output interface 215, alternative embodiments of the electronic controller 33 may include multiple processors, memory modules, and/or input/output interfaces. It should also be noted that the system 10 may include other electronic controllers, each including similar components as, and configured similarly to, the electronic controller 33. In some embodiments, the electronic controller 33 is implemented partially or entirely on a semiconductor (for example, a field-programmable gate array [“FPGA”] semiconductor) chip. Similarly, the various modules and controllers described herein may be implemented as individual controllers, as illustrated, or as components of a single controller. In some aspects, a combination of approaches may be used.

As described above, the catheter 12 includes the LED 44. The electronic controller 33 (in particular, the electronic processor 205) is configured to operate the LED 44. As also described above, in some aspects the controller 33 operates the LED 44 based on a user input received via the input/output interface 215. Alternatively or additionally, in some aspects the controller 33 operates the LED 44 according to information from one or more sensors of the catheter 12 (for example, the temperature sensor 30 and/or the pressure sensor(s) 42).

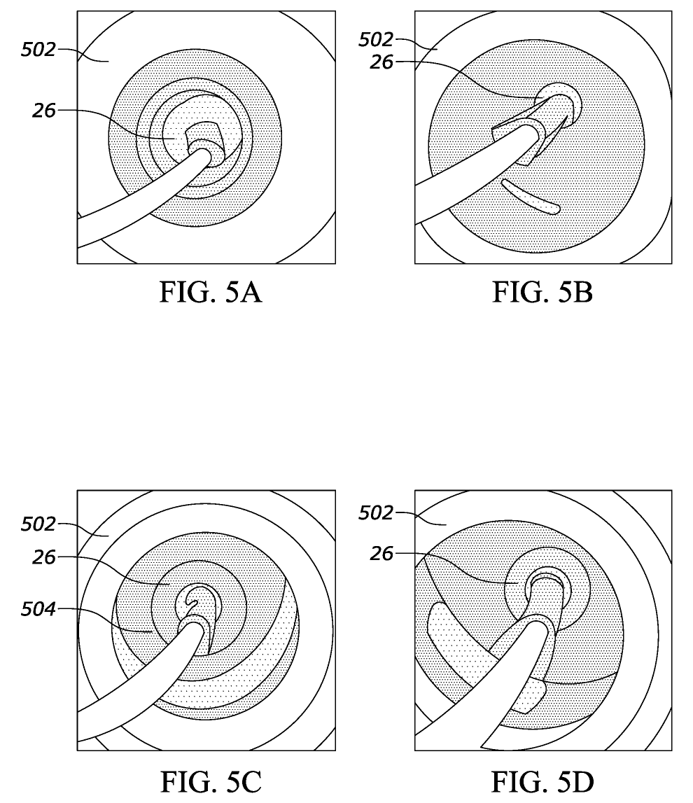

The LED 44 may be operated as a visual aid for a user of the catheter 12 to verify full circumferential contact (physical contact along a complete circumference) of the expandable element 26 within a lumen (for example, a bronchus) of a patient. For example, FIGS. 5A – 5D each illustrate the expandable element 26 at a plurality of different stages of inflation/circumferential within an example lumen 502. In the examples illustrated in FIGS. 5A – 5D, the LED 44 is disposed at the distal portion 22 between the guidewire 31 and the expandable element 26. FIG. 5A is an example instance where the expandable element 26 is not in contact with any surface within the lumen 502. FIG. 5B is an example instance where the expandable element 26 is partially expanded and in partial contact with an inner surface within the lumen 502. FIG. 5C is an example instance where the expandable element 26 is partially expanded and in partial contact with the inner surface within the lumen 502 with a portion of the element 26 not in contact with the inner surface (gap 504). FIG. 5D is an example instance where the expandable element 26 is expanded and in full circumferential contact with the inner surface within the lumen 502.

In instances where a user of the catheter 12 visually (with visual aid from the LED 44) determines that contact of the expandable element 26 within a lumen of the patient is not complete, as described above, the user may be prompted to adjust a position of the catheter 12 and/or adjust a diameter of (for example, by adjusting a pressure and/or amount of fluid within) the expandable element 26.

In some aspects, the electronic controller 33 controls power to the LED 44 via a wired connection that also provides power to at least one of the other sensors (for example, the temperature sensor 30 and/or the pressure sensor(s) 42) within the distal portion. In some instances, the electronic controller 33 may be configured to operate the LED 44 for a predetermined interval/at a predetermined duty cycle and, between operations of the LED 44 (for example, when the LED 44 is off), provide power instead to (and/or receive/record measurements from) the sensor of the shared wired connection. For example, in instances where the LED 44 and the temperature sensor 42 share a wired connection, the electronic controller 33 may be configured to alternatively power/perform measurements with the temperature sensor 42 and the LED 44 for a predetermined period (for example, one second). In some aspects, power is additionally or alternatively provided to the LED 44 via one or more other conductive elements of the catheter 12 (for example, via a conductive trace (not shown) of/within the elongate segment 18, the treatment element 32, and/or the inflow lumen 27).

In some aspects, the LED 44, as described above, is disposed within the handle 24 of the catheter 12 and is aimed at the expandable element 26. In such aspects, an inside of the elongate body 18 may be coated in a reflective inner coating (for example, to aid in concentrating light to the expandable element 26).

In some aspects, the exterior of the expandable element 26 may include an impedance, capacitive, or resistive-based coating in a circumferential pattern. In some aspects, the exterior of the expandable element 26 may include a plurality of discrete sensors in a circumferential pattern.

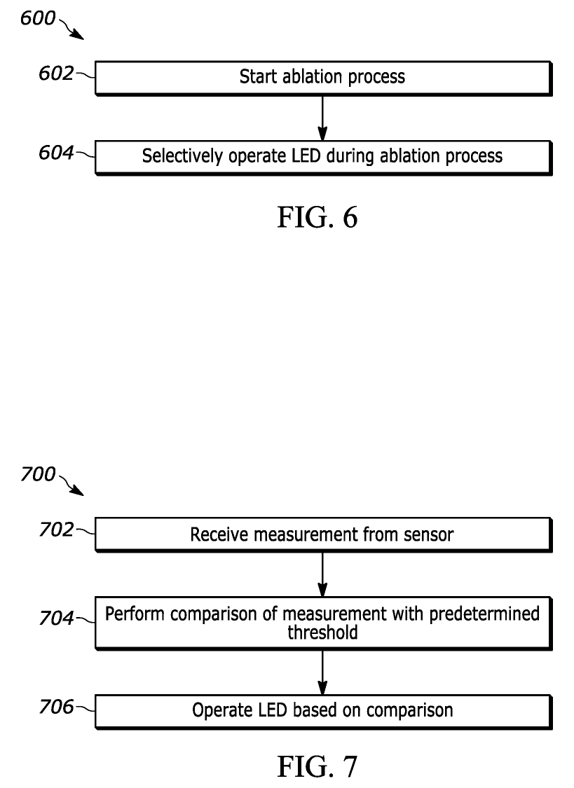

FIG. 6 illustrates an example method 600 for operating the system of FIG. 1 to operate the catheter 12 and provide visual aid via operation of the LED 44 during ablation procedures in accordance with some aspects. Although the method 600 is described in conjunction with the system 10 as described herein, the method 600 could be used with other systems and devices. In addition, the method 600 may be modified or performed differently than the example provided.

As an example, the method 600 is described as being performed by the electronic controller 33. However, it should be understood that, in some examples, portions of the method 600 may be performed by other components, including for example, the catheter 12.

At block 602, the electronic controller 33 initiates an ablation process, for example, by controlling the fluid source 16 to provide a volume of the fluid to the expandable element 26 via the inflow lumen 27. In one aspect, the electronic controller 33 controls the vacuum pump 34 or a similar pump to provide a volume of refrigerant to the expandable element 26. In another aspect, the electronic controller 33 controls a valve or valves (not shown) to provide a volume of refrigerant to the expandable element 26 from a pressurized source. It should be understood that the term “ablation process” used herein refers to inflation and deflation of the expandable element 26 and includes instances where the element 26 is not actively ablating a target.

At block 604, the electronic controller 33 selectively operates the LED 44 during the ablation process. Operating the LED 44 may include, for example, changing an intensity of the LED 44 and/or changing a duty cycle of the LED 44. In some aspects, the LED 44 is a multi-color LED. In such aspects, operating the LED 44 may include changing a color of the LED 44. In some aspects, the electronic controller 33 selectively operates the LED 44 based on a user input (received, for example, via the input/output interface 215). In implementations where there are multiple LEDs 44, for example, a user may provide a command to the controller 33 (via the input/output interface 215) to select which LEDs 44 to activate and set operational characteristics for each LED 44 (for example, duty cycle, intensity, color, etc.).

In some aspects, the electronic controller 33 is configured to automatically control operation of the LED 44 based on one or more measurements from one or more sensors (for example, the temperature sensor 30 and/or the pressure sensor(s) 42). For example, FIG. 7 is a flowchart of a method 700 implemented by the electronic controller 33 for selectively operating the LED 44 (block 604 of the method 600 of FIG. 6) in accordance with some aspects.

At block 702, the electronic controller 33 receives a measurement from a sensor configured to measure a characteristic of the treatment element 23 (for example, the temperature sensor 30 and/or the pressure sensor(s) 42). In some aspects, the characteristic is a temperature or a pressure of fluid within the treatment element 23. In some aspects, the characteristic is a pressure of fluid within the expandable element 23.

At block 704, the electronic controller 33 performs a comparison of the measurement with a predetermined threshold and, at block 706, operates the LED 44 based on the comparison. The predetermined threshold may be a maximum threshold or a minimum threshold. In some aspects, the electronic controller 33 compares the measurement to more than one predetermined threshold (for example, to determine whether the measurement falls within a range defined by two different predetermined thresholds. The electronic controller 33, based on the comparison, operates the LED 44 (for example, duty cycle, intensity, color, etc.). For example, in instances where the comparison indicates an abnormality of the treatment element 23 (for example, the measurement is outside of an acceptable range, is above a maximum threshold, or is below a minimum threshold), the electronic controller 33 operates the LED 44 to provide a visual alert to the user of the catheter 12 indicative of the abnormality (while still operating the LED 44 during the ablation process to provide visual aid to the user).

In the foregoing specification, specific examples have been described. However, one of ordinary skill in the art appreciates that various modifications and changes can be made without departing from the scope of the invention as set forth in the claims below. Accordingly, the specification and figures are to be regarded in an illustrative rather than a restrictive sense, and all such modifications are intended to be included within the scope of present teachings.

Moreover, in this document, relational terms such as first and second, top and bottom, and the like may be used solely to distinguish one entity or action from another entity or action without necessarily requiring or implying any actual such relationship or order between such entities or actions. The terms “comprises,” “comprising,” “has,” “having,” “includes,” “including,” “contains,” “containing,” or any other variation thereof, are intended to cover a non-exclusive inclusion, such that a process, method, article, or apparatus that comprises, has, includes, contains a list of elements does not include only those elements but may include other elements not expressly listed or inherent to such process, method, article, or apparatus. An element proceeded by “comprises …a,” “has …a,” “includes …a,” or “contains …a” does not, without more constraints, preclude the existence of additional identical elements in the process, method, article, or apparatus that comprises, has, includes, contains the element. The terms “a” and “an” are defined as one or more unless explicitly stated otherwise herein. The terms “substantially,” “essentially,” “approximately,” “about,” or any other version thereof, are defined as being close to as understood by one of ordinary skill in the art, and in one non-limiting embodiment the term is defined to be within 10%, in another embodiment within 5%, in another embodiment within 1% and in another embodiment within 0.5%. The term “coupled” as used herein is defined as connected, although not necessarily directly and not necessarily mechanically. A device or structure that is “configured” in a certain way is configured in at least that way but may also be configured in ways that are not listed.

It will be appreciated that some examples may be comprised of one or more generic or specialized processors (or “processing devices”) such as microprocessors, digital signal processors, customized processors and field programmable gate arrays (FPGAs) and unique stored program instructions (including both software and firmware) that control the one or more processors to implement, in conjunction with certain non-processor circuits, some, most, or all of the functions of the method and/or apparatus described herein. Alternatively, some or all functions could be implemented by a state machine that has no stored program instructions, or in one or more application specific integrated circuits (ASICs), in which each function or some combinations of certain of the functions are implemented as custom logic. Of course, a combination of the two approaches could be used.

Moreover, an embodiment can be implemented as a computer-readable storage medium having computer readable code stored thereon for programming a computer (e.g., comprising a processor) to perform a method as described and claimed herein. Examples of such computer-readable storage mediums include, but are not limited to, a hard disk, a CD-ROM, an optical storage device, a magnetic storage device, a ROM (Read Only Memory), a PROM (Programmable Read Only Memory), an EPROM (Erasable Programmable Read Only Memory), an EEPROM (Electrically Erasable Programmable Read Only Memory) and a Flash memory. Further, it is expected that one of ordinary skill, notwithstanding possibly significant effort and many design choices motivated by, for example, available time, current technology, and economic considerations, when guided by the concepts and principles disclosed herein will be readily capable of generating such software instructions and programs and ICs with minimal experimentation.

The following paragraphs provide various examples and alternatives of the embodiments disclosed herein.

Example 1. A system for ablating tissue, the system comprising: a catheter including a treatment element, the treatment element including an expandable element; a light emitting device positioned and configured to emit light at the expandable element; and an electronic controller coupled to the catheter and configured to: initiate an ablation process by controlling a fluid source to provide fluid to the expandable element of the treatment element; and selectively operate the LED during the ablation process.

Example 2. The system of clause 1, the system further including a sensor configured to measure a characteristic of the treatment element and wherein the electronic controller is further configured to: receive a measurement from the sensor; perform a comparison of the measurement to a predetermined threshold; and operate the light emitting device based on the comparison.

Example 3. The system of clause 2, wherein operating the light emitting device based on the comparison includes changing a color of the light emitting device.

Example 4. The system of clause 2, wherein operating the light emitting device based on the comparison includes changing a duty cycle of the light emitting device.

Example 5. The system of clause 2, wherein operating the light emitting device based on the comparison includes changing an intensity of light from the light emitting device.

Example 6. The system of clause 2, wherein the characteristic of the treatment element is a temperature of the fluid in the treatment element.

Example 7. The system of clause 2, wherein the characteristic of the treatment element is a pressure of the fluid in the treatment element.

Example 8. The system of clause 7, wherein the characteristic of the treatment element is a pressure within the expandable element.

Example 9. The system of clause 1, wherein the light emitting device is disposed at a distal portion of the treatment element at a distal end of the expandable element.

Example 10. The system of clause 1, wherein the light emitting device is disposed on a handle of the catheter.

Example 11. A method for operating an ablation catheter including a treatment element including an expandable element and a light emitting device positioned and configured to emit light at the expandable element, the method comprising: initiating an ablation process by controlling a fluid source to provide fluid to the expandable element of the treatment element; and selectively operate the light emitting device during the ablation process.

Example 12. The method of clause 11 further including: receiving a measurement from a sensor configured to measure a characteristic of the treatment element; performing a comparison of the measurement to a predetermined threshold; and operating the light emitting device based on the comparison.

Example 13. The method of clause 12, wherein operating the light emitting device based on the comparison includes changing a color of the light emitting device.

Example 14. The method of clause 12, wherein operating the light emitting device based on the comparison includes changing a duty cycle of the light emitting device.

Example 15. The method of clause 12, wherein operating the light emitting device based on the comparison includes changing an intensity of light from the light emitting device.

Example 16. The method of clause 12, wherein the characteristic of the treatment element is a temperature of the fluid in the treatment element.

Example 17. The method of clause 12, wherein the characteristic of the treatment element is a pressure of the fluid in the treatment element.

Example 18. The method of clause 12, wherein the characteristic of the treatment element is a pressure within the expandable element.

Example 19. The method of clause 11, wherein the light emitting device is disposed at a distal portion of the treatment element at a distal end of the expandable element.

Example 20. The method of clause 11, wherein the light emitting device is disposed on a handle of the catheter.

Various features and advantages of the embodiments presented herein are set forth in the following claims.

What is claimed is:

1. A system for ablating tissue, the system comprising:
a catheter including a treatment element, the treatment element including an expandable element;
a light emitting device positioned and configured to emit light at the expandable element; and
an electronic controller coupled to the catheter and configured to:
initiate an ablation process by controlling a fluid source to provide fluid to the expandable element of the treatment element; and
selectively operate the LED during the ablation process.

2. The system of claim 1, the system further including a sensor configured to measure a characteristic of the treatment element and wherein the electronic controller is further configured to:
receive a measurement from the sensor;
perform a comparison of the measurement to a predetermined threshold; and
operate the light emitting device based on the comparison.

3. The system of claim 2, wherein operating the light emitting device based on the comparison includes changing a color of the light emitting device.

4. The system of claim 2, wherein operating the light emitting device based on the comparison includes changing a duty cycle of the light emitting device.

5. The system of claim 2, wherein operating the light emitting device based on the comparison includes changing an intensity of light from the light emitting device.

6. The system of claim 2, wherein the characteristic of the treatment element is a temperature of the fluid in the treatment element.

7. The system of claim 2, wherein the characteristic of the treatment element is a pressure of the fluid in the treatment element.

8. The system of claim 7, wherein the characteristic of the treatment element is a pressure within the expandable element.

9. The system of claim 1, wherein the light emitting device is disposed at a distal portion of the treatment element at a distal end of the expandable element.

10. The system of claim 1, wherein the light emitting device is disposed on a handle of the catheter.

11. A method for operating an ablation catheter including a treatment element including an expandable element and a light emitting device positioned and configured to emit light at the expandable element, the method comprising:
initiating an ablation process by controlling a fluid source to provide fluid to the expandable element of the treatment element; and
selectively operate the light emitting device during the ablation process.

12. The method of claim 11 further including:
receiving a measurement from a sensor configured to measure a characteristic of the treatment element;
performing a comparison of the measurement to a predetermined threshold; and
operating the light emitting device based on the comparison.

13. The method of claim 12, wherein operating the light emitting device based on the comparison includes changing a color of the light emitting device.

14. The method of claim 12, wherein operating the light emitting device based on the comparison includes changing a duty cycle of the light emitting device.

15. The method of claim 12, wherein operating the light emitting device based on the comparison includes changing an intensity of light from the light emitting device.

16. The method of claim 12, wherein the characteristic of the treatment element is a temperature of the fluid in the treatment element.

17. The method of claim 12, wherein the characteristic of the treatment element is a pressure of the fluid in the treatment element.

18. The method of claim 12, wherein the characteristic of the treatment element is a pressure within the expandable element.

19. The method of claim 11, wherein the light emitting device is disposed at a distal portion of the treatment element at a distal end of the expandable element.

20. The method of claim 11, wherein the light emitting device is disposed on a handle of the catheter.
